(19) United States
(12) Patent Application Publication
(10) Pub. No.: US 2016/0150963 A1
(43) Pub. Date: Jun. 2, 2016

(54) ONE-PHOTON INTEGRATED NEUROPHOTONIC SYSTEMS

(71) Applicants: Michael Lee Roukes, Pasadena, CA (US); Ronald James Cotton, Houston, TX (US); Laurent Moreaux, Pasadena, CA (US); Kenneth Shepard, Ossining, NY (US); Athanassios Siapas, Pasadena, CA (US); Andreas Tolias, Houston, TX (US)

(72) Inventors: Michael Lee Roukes, Pasadena, CA (US); Ronald James Cotton, Houston, TX (US); Laurent Moreaux, Pasadena, CA (US); Kenneth Shepard, Ossining, NY (US); Athanassios Siapas, Pasadena, CA (US); Andreas Tolias, Houston, TX (US)

(21) Appl. No.: 14/534,163

(22) Filed: Nov. 5, 2014

Related U.S. Application Data
(63) Continuation-in-part of application No. 13/627,755, filed on Sep. 26, 2012.
(60) Provisional application No. 62/054,893, filed on Sep. 24, 2014.
(60) Provisional application No. 61/900,216, filed on Nov. 5, 2013.
(60) Provisional application No. 61/539,133, filed on Sep. 26, 2011.
(60) Provisional application No. 61/568,331, filed on Dec. 8, 2011.

(51) Int. Cl. A61B 5/00

(52) U.S. Cl. CPC A61B 5/0071; A61B 5/4064; A61B 5/4029

(57) ABSTRACT

An apparatus and method for detecting functional cellular activity within a volume of a tissue. The method includes inserting a three-dimensional array of optical emitters and optical detectors into a volume of a tissue, where the tissue volume includes one or more cells labeled with an optical reporter of cellular activity; illuminating the one or more cells with photons from the optical emitters of the three-dimensional array to generate optical signals from the optical reporter that labels the one or more cells; and detecting the optical signals using the optical detectors of the three-dimensional array, where the illumination includes one-photon excitation of the optical reporter.

### BACKGROUND

1. Field of the Invention

The invention relates to an apparatus and method for functional imaging of tissue.

2. Related Art

Over the past few decades, our understanding of the properties of individual neurons and their role in brain computations has advanced significantly. However, we are still very far from understanding how large ensembles of neurons in the brain interact to process information. For monitoring neuronal activity, extracellular electrical recording provides unparalleled temporal resolution. It is not possible, though, to record electrically from specific cell types, and up-scaling recording density to track the activity of every neuron in an extended brain region appears infeasible. Functional imaging by free-space two-photon microscopy enables single-cell resolution of large neuron ensembles at anatomical densities and provides cell-type specificity of activity via genetically encoded fluorescent reporters. But it works ideally only with thin and transparent specimens. More generally, light scattering and absorption in tissue impose significant fundamental limits: in mammalian brains, accessible depths in vivo are restricted to superficial cortical regions, <1 mm. Endoscopic methods developed to circumvent such restrictions impart significant damage to tissue given the large probe diameter (0.3 to >1 mm).

More than a century ago, Ramon y Cajal speculated that the brain's varied and complex functions arise from two fundamental properties of neurons: their individual morphologies, and their connections to each other. A modern revision of these precepts underlies the current perspective on the cerebral cortex: first, different regions of the brain contain distinct, genetically specified neuronal cell types—and these cell types possess distinct and characteristic electrophysiological morphological properties (i.e. dendritic inputs, axonal outputs); and second, this variety of cell types seem to be arranged in stereotypical microcircuits that enable each brain area's local functions. As have neuroscientists since Cajal, it is presently believed that the key to understanding how the brain works is first to attain an understanding of how different neuron classes interact functionally, in vivo. This detailed knowledge is expected to elucidate how functional, i.e. microcircuit, interactions break down in disease. At present, the requisite tools to monitor complex brain circuits do not exist, however, and this has posed a universal and long-standing obstacle to such pursuits.

### SUMMARY

In some aspects, a new approach of integrated neurophotonics is provided. Integrated neurophotonics is a novel paradigm for functional optical imaging that surmounts the limits of present methods. It permits functional imaging with cellular resolution in highly scattering brain tissue, can offer complete coverage of all neurons within target volumes, and has eventual prospects for human applications. This approach is based on distributing a dense 3-D lattice of emitter and detector pixels within the brain itself, spaced by distances on the order or less than the optical attenuation length. These pixel arrays are embedded onto neurophotonic probes, realized as implantable, ultra narrow shanks that leverage recent advances in nanoprobe-based electrophysiology and integrated nanophotonics. Used with functional optical reporters, one 25-shank probe module is capable of recording activity from all neurons within a 1-mm³ volume (˜100,000 neurons). Further, this methodology is scalable; multiple modules can be tiled to densely cover extended regions deep within the brain. Accordingly, it can permit simultaneous recording from millions of neurons, at arbitrary positions and depths in the brain, to unveil dynamics of complete neural networks—with single-cell resolution and cell-type specificity. Ultra narrow neurophotonic probes can perturb brain tissue minimally, and can impose negligible tissue displacement and only minute local power dissipation. Importantly, the neurophotonic probes can be produced by existing methods of large-scale integration via wafer-scale foundry (factory) based technology. The probes will transform studies of circuit-level mechanisms of brain computation and neurological disorders, and accelerate drug discovery by high throughput screening in vivo.

In one aspect, a method for detecting functional cellular activity within a volume of a tissue is provided. The method includes, a) inserting a three-dimensional array of optical emitters and optical detectors into a volume of a tissue, the tissue volume including one or more cells labeled with an optical reporter of cellular activity, b) illuminating the one or more cells with photons from the optical emitters of the three-dimensional array to generate optical signals from the optical reporter that labels the one or more cells, and c) detecting the optical signals using the optical detectors of the three-dimensional array, wherein the illumination includes one-photon excitation of the optical reporter.

In embodiments of the method, a) the optical signals are fluorescent optical signals, b) the tissue is nervous tissue or living brain tissue, and each cell is labeled with an optical reporter of neural activity, c) the optical reporter is a genetically encoded fluorescent protein, a chemical fluorescent reporter, or a fluorescent nanoparticle reporter, or a combination thereof, d) the array includes elongated microsized shanks including the optical emitters and the optical detectors, each shank being 100 μm or less in width, e) each shank includes optical emitters and optical detectors, f) the shanks extend to any arbitrary location in the tissue, g) the optical emitters are time-gated, h) the optical emitters include optical elements for spatial profile control of the illumination, i) the optical detectors include optical filters, focusing elements, planar optical elements, or metamaterial-based optical elements, or any combination thereof, j) the detection elements includes both continuous or time-gated collection of the optical signals, k) the detection includes optical intensity sensing of the optical signals, or optical signal detection by avalanche current amplification of individual photon absorption events, or any combination thereof, l) the method further includes optical spike sorting of the detected optical signals, or m) any combination of a)-l).

In another aspect, a device for detecting functional cellular activity is provided. The device includes, elongated microsized shanks, each shank including one or more optical emitters and one or more optical detectors, wherein the shanks are sized in width and thickness to fit between adjacent neuronal cell bodies in a neural tissue, and the shanks are arranged to form a three-dimensional array of the optical emitters and the optical detectors.

In embodiments of the device, a) the shanks are about 100 μm or less in width, b) the shanks are about 1 mm or more in length, c) the array has a pitch that is on the order of, or less than, one optical attenuation length of a predetermined wavelength of light to be emitted from the optical emitters, d) the optical detectors include optical filters, or focusing elements, or any combination thereof, e) the emitters are time-gated, f) the optical detectors include optical intensity sensors or avalanche current amplification sensors, g) the device further includes a time-to-digital converter connected to the optical detectors

### BRIEF DESCRIPTION OF THE DRAWINGS

For a more complete understanding of the present invention, reference is now made to the following descriptions taken in conjunction with the accompanying drawings, in which:

FIG. 1 is a comparison of functional imaging by free-space optics versus by integrated neurophotonic probes. 1a) The present state-of-the-art in free-space, calcium-based functional imaging employs fluorescence microscopy based on two-photon excitation. However, even under favorable conditions with near infrared illumination, only superficial depths (˜1 mm) are accessible for functional imaging due to the short optical attenuation length in brain tissue, L_{A}, arising from fundamental absorption and scattering processes. (Data adapted from C. Xu, Cornell University.) 1b) The concept underlying the inventors' approach is illustrated by comparing decay of the ballistic trajectories for the excitation (red) and emission (green). Emitter and detector pixels (“E-pixels” and “D-pixels”, respectively) are labeled. With free-space optics, depicted in the left panel, interrogation depth is limited to several times L_{A}. With photonic probes (right panel) emitters and detectors are always within ˜L_{A} of the object under study—enables functional imaging at arbitrary depths. 1c) The integrated neurophotonics paradigm places a multiplicity of light sources and detectors, separated by less than an optical attenuation length, at arbitrary depths and positions within the brain.

FIG. 2 is an illustration of a state-of-the-art two-photon microscope for functional imaging. 2a) Layout of the 3-D random-access multiphoton (3D-RAMP) microscope [4]. The expanded beam of a pulsed Ti: Sapphire laser is shaped in collimation and angle at the back focal plane of the objective lens by a chain of four acousto-optic deflectors (AODs) and telescopes; this yields 3-D positioned focus. 2b) Recording from a population of neurons in the visual cortex of an awake, head-fixed mouse with the 3D-RAMP setup. All 411 cell bodies in a 200×200×100 μm³ volume are located. Neurons traces shown in c) are labeled green. Ca activity from 411 neurons are sampled at 122 Hz. 2c) Calcium traces of cells in volume in 2b) are labeled in green. Functional traces are down-sampled from 122 Hz to 20 Hz for visualization and comparison.

FIG. 3 is an panel showing the neurophotonic probe paradigm. 3a) Conceptual schematic of 25-shank module, implanted several mm deep to enable functional imaging of all neurons within a 1 mm³ volume of the hippocampus. 3b) On each probe shank are embedded 80 photon-counting detectors (green rectangles) and 80 waveguide emitters (red lines). The 25 μm wide shanks are comparable to the size of the cell bodies, as shown in the figure. 3c) Micrograph of a typical single photon avalanche photodetector (SPAD) Below is shown cross-sections of the layer structure, and the simulated electronic profile. d) Micrograph of a time-to-digital-converter (TDC) circuit, atypically separated from the detectors and located at the top of the photonic probes, to minimize heat delivery to brain tissue.

FIG. 4 is a panel of micrographs showing nanoprobes for electrophysiology fabricated at Caltech in 2013. 4a) Wafer scale nanofabrication of prototype nanoprobes created within Caltech's Kavli Nanoscience Institute. These are compatible with micro-electronics-foundry-based production; efforts are underway to fabrication en masse at the 200 mm wafer scale. 4b,4c,4d) A variety of designs permit realization of probes optimized for recording in different regions of the brain. 4e) Magnified view of the 128 μm² recording sites, and their nanoscale connection traces. 4f) A module comprising a stack of four nanoprobe layers, which constitutes a 3-D array for recording with 1024 sites.

FIG. 5 is a illustration showing how neurophotonic probes can record the activity from one unit volume of neural tissue. 5a) The prototype geometry depicted comprises five, 5-shank layers to create a 25-shank array. The probes are on a (rotated) square grid with 283 μm sides. This, and the 50 μm pitch of the E- and D-pixels on the shanks, delineate a unit volume of 0.004 mm³, which contains ˜400 neurons at the typical mouse cortex density. 5b) Conceptual top view of one unit volume; an optical micrograph of labeled neurons of the mouse cortex is superimposed for scale. Emitter pixels at the top and bottom illuminate neurons within (blue arrows); more complex illumination patterns are possible with the 18 E-pixels that are within one attenuation length from the target neuron. Similarly, photons emitted during a neuron's fluorescence (green) are collected by the 18 D-pixels within one attenuation length. Together they provide a high-dimensional measurement space enabling optical spike sorting, as described herein.

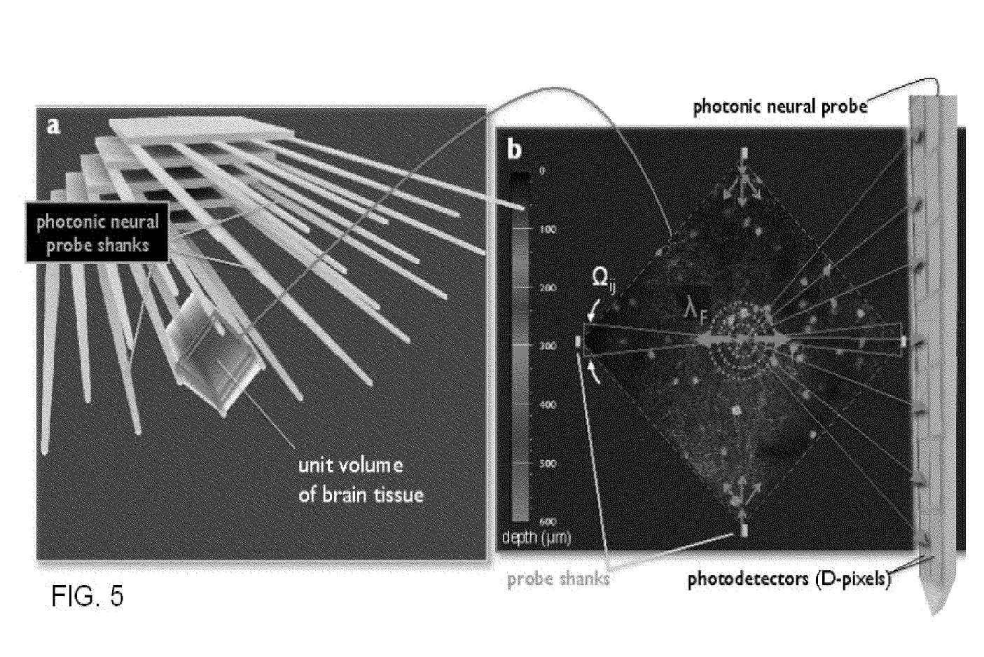

FIG. 6 is an illustration showing an example schematic configuration enabling SPAD operation and ancillary circuitry on which the D-pixels can be based. 6a) Structural cross section of device showing a single-photon absorption event triggering a carrier avalanche. (inset). The impulse response of the SPAD from Ref. 32 as recorded by its on-chip TDCs is 125 ps. Each bar in the histogram represents a 62.5 ps wide timing bin. 6b) Schematic of the pixel-level circuit that performs the quench, reset, TDC calibration, event output, and other control functions.

FIG. 7 is an illustration providing a conceptual picture of the principles that underlie the methodology optical spike sorting. 7a) The fluorescence of N labeled neurons within a unit volume is excited by patterned illumination from an array of n E-pixels (blue arrows). Their emission is recorded by an array of n D-pixels (green). 7b) Optical spike sorting is the “de-mixing” process by which data from the “measurement space” is used to compute the individual fluorescence time records for the N neurons involved; i.e. its conversion to the “data space”. As described, the measurement space is sufficiently complex to permit such a transformation.

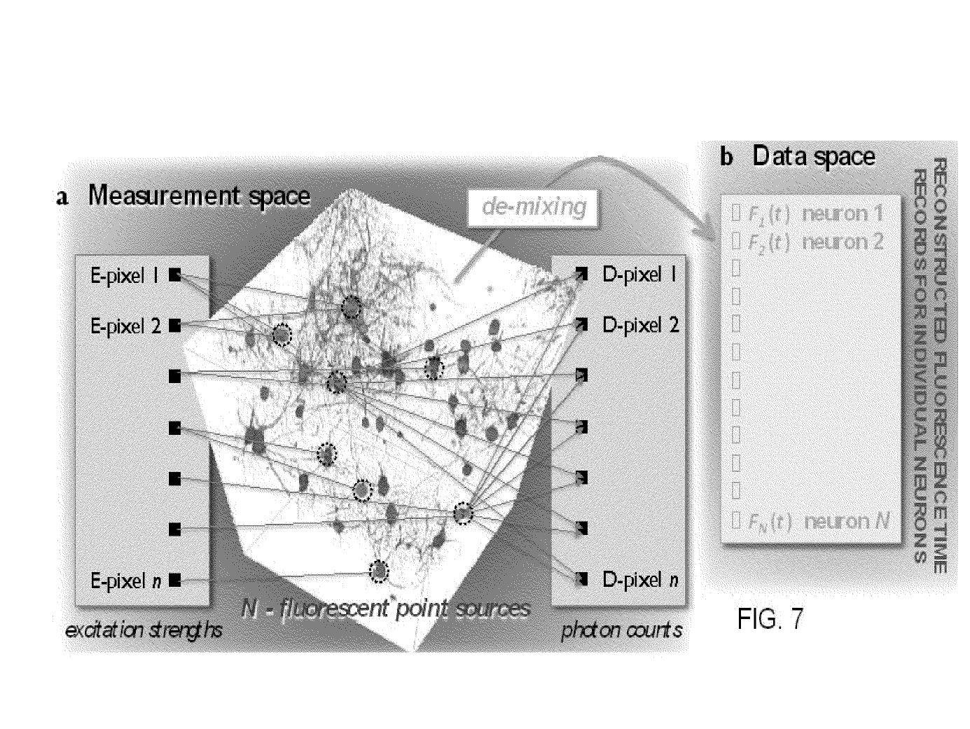

FIG. 8 is an illustration showing the results of simulations of optical spike sorting. To validate the concept of optical spike sorting for photonic probes a simulation of 360 neurons randomly positioned within a 282×282×50 μm³ unit volume is carried out. First, the effectiveness of each emitter (E, blue dots) for inducing fluorescence of the labeled neurons is calculated. Then, the photon collection efficiency for each detector (D, green dots) is determined for each fluorescing neuron. An optical attenuation length L_{A}=200 μm is assumed. The graph inset shows the distribution of minimum Mahalanobis distances across the neuronal population. The median Mahalanobis distance is 17 with a 5% lower quantile of 7.1 and 95% upper quantile of 34. These values indicate excellent segregation of the individual optically reported calcium events, triangulated to the appropriate neuron. Hence, calcium events are classifiable to their source neuron throughout the volume, not only near the probes.

FIG. 9 is a drawing of a neuron within neural tissue prepared with functional optical reporters.

FIG. 10 is a drawing showing co-integration of emitter and detector pixels on the same shank.

FIG. 11A is a drawing showing an embodiment of the multi-layer assembly of a probe ensemble including a probe with 25 shanks in a 5-layer assembly with five shanks per layer.

FIG. 11B illustrates the terminal end of a single shank.

FIG. 12 illustrates the photon-counting mode of operation for a single-photon avalanche photodiode (SPAD).

FIG. 13 is a block diagram of a full architecture of an embodiment of an integrated neurophotonics data acquisition system.

### DETAILED DESCRIPTION

The following are incorporated by reference herein: U.S. Provisional Patent Application Nos. 61/900,216, filed on Nov. 5, 2013, and 62/054,893, filed on Sep. 24, 2014, and U.S. patent application Ser. No. 13/627,755, filed on Sep. 26, 2012.

In a particular aspect, a method for detecting functional cellular activity within a volume of a tissue is provided. In the method, a three-dimensional array of optical emitters and optical detectors is inserted into the tissue volume. The array can be realized as a probe having elongated microsized shanks of an arbitrary length to reach any region within the tissue, with the shanks comprising optical emitters and/or optical detectors. In some embodiments, a shank can have a length of about 1 mm or more, about 2 mm or more, about 3 mm, about 4 mm or more, or about 5 mm or more. A shank can be sized to minimize damage to the tissue. For example, a shank can be sufficiently narrow so as to circumvent immune responses, scarring and gliosis after implantation into brain or other nervous tissue. In some embodiments, the width of the shank can be about 500 μm or less, about 400 μm or less, about 300 μm or less, about 200 μm or less, about 100 μm or less, about 50 μm or less, or about 25 μm or less, and the thickness of the shank can be about 100 μm or less, about 75 μm or less, about 50 μm or less, about 25 μm or less, or about 15 μm or less. Different shanks in an array can have different lengths, widths and/or thicknesses than other shanks in the array, and some or all of the shanks in an array can be similarly sized.

To form the array, the microsized shanks can be arranged to form a three-dimensional array of shanks. In some embodiments, the shanks can be ultra-thin and ultra-narrow shanks, giving the array of shanks a small total cross-sectional area (transverse to the length of the shanks) that minimizes the displacement of, and perturbation to, the tissue.

Examples of optical emitters include, but are not limited to, waveguide terminals, micro-ring resonators, photonic crystal resonators, microfabricated diffraction gratings, nano-fabricated pillars, or zone-plates, or any combination thereof.

Examples of time-gated semiconductor-based optical detectors include, but are not limited to, photon-counting detectors, such as single-photon avalanche photo-detectors (SPADs); or integrating detectors; with internal gain, such as avalanche photodiodes, or without, such as PIN photodiodes; or any combination thereof.

The pitch of the three-dimensional array of optical emitters and photo-detectors can be adjusted by changing either or both the shank-to-shank spacing, and the spacing of the optical emitter and detector elements upon each shank. The pitch can be chosen to scale with the optical attenuation length of the neural tissue at the wavelength(s) employed by the functional optical reporters. The choice of pitch permits the system's optical emitters and detectors to operate near or within the regime of ballistic photon propagation.

In some embodiments, the photodetector elements can include optical filters, focusing elements, planar optical elements, or metamaterial-based optical elements, or any combination thereof. For example, the photodetector elements can include spectral filters that enable optical signals from the functional optical reporters to be separated from other undesired sources of illumination (which can include endogenous tissue fluorescence by pigments absorption). Examples of spectral filtering components include, but are not limited to, resonant cavities, gratings, nanopillars or other nanostructures, or plasmonic absorption elements, or combinations thereof. In addition, the photodetector elements can include focusing elements such as microlens elements, which can enhance the collection of illumination emanating from functional optical reporters.

Cells in the tissue volume can be labeled with an optical reporter of cellular activity, including an optical reporter of functional neural activity. Examples of optical reporters, include but are not limited to: a) genetically encoded fluorescent proteins that report neural activity, including voltage indicators, chemical indicators such as calcium, pH, neuromodulator or neurotransmitter indicators, indicators sensitive to local forces, etc.; b) exogenous fluorescent activity reporters, for example, chemically-sensitive fluorescent molecules or nanoparticles, such as calcium-sensitive reporters like GCaMP; c) fluorescent voltage-sensitive dyes or nanoparticles, or other reporters of neural activity, including voltage indicators, chemical indicators such as calcium, pH, neuromodulator or neurotransmitter indicators, or indicators sensitive to local forces; d) or any combination thereof (see, for example, Molecular Probes-Production Information MP03010, Long-Wavelengh Calcium Indicators (2005); C. Grienberger, A. Konnerth, Imaging calcium in neurons. Neuron 73, 862-885 (2012); J. Akerboom, et al, Optimization of a GCaMP calcium indicator for neural activity imaging. Journal of Neuroscience 32, 13819-13840 (2012); all incorporated by reference herein).

In some embodiments, the optical emitter arrays on the shanks can deliver programmed, sub-nanosecond pulsed-excitation light within the tissue, which can be neural tissue, with repetitive or asynchronous rates engineered to permit optimal signal extraction defined by the properties of the functional optical reporters. In these and other embodiments, the optical detector arrays on the shanks can permit programmed signal integration (including either intensity integration or photon counting) of the time-varying illumination that is impingent upon them. Programmed operation can include time-gated collection that permits rejection of the “feed-through” illumination (which results from, and occurs during, excitation pulses), allowing it to be separated from the desired, functionally-induced optical signals emanating from the optical reporters.

Integrated neurophotonics is a novel technology that enables unprecedentedly dense, simultaneous, and cell-type specific monitoring of neurons and their interactions, in vivo, in real time. As an example, elucidating the neural circuitry of the neocortex is among the new classes of studies possible—for this, recording neural activity with cellular resolution and cell-type specificity in all six cortical layers will be required. Cortical architecture appears to be organized in columns; in the mouse brain these contain ˜100,000 neurons in a ˜1-mm³ volume. The fact that ˜90% of the column's connections are local[1,2] suggests detailed investigation of these as candidate microcircuits. Here, to clarify description of the technology and to provide concrete methods for its embodiment, the specific target of recording densely from a single cortical column will be used as an example.

One embodiment includes the recording of all the activity from the ˜100,000 neurons within one cortical column of a mouse. The system can be modular and scalable; this permits tiling multiple nanophotonic modules to cover neural circuits spanning extended brain regions. Engaging in large-scale production of integrated neurophotonic modules can make it feasible to enable recording from millions of neurons with single-neuron resolution. For example, assembly of ten of the prototype modules described herein would enable recording from all ˜1 million neurons in the mouse visual cortex.

The research enabled by these powerful tools will provide unprecedented and massive data sets that will, in turn, enable a mechanistic understanding of how the cortical circuits functions normally and how they fail in neuropsychiatric disorders. Recording from all neurons in a local circuit will revolutionize understanding of information processing in the brain. For example, it would enable testing of the long-standing idea that the neocortex is built from repeating computational circuit modules. By contrast, present in vivo 3D-imaging technologies are many orders magnitudes away from being able to achieve such a result.

Integrated neurophotonics will ultimately also transform the study of neuropsychiatric disorders such as depression and post-traumatic stress disorder (PTSD). These devastating illnesses are believed by many to be brain circuit dysfunctions resulting from subtle alterations of circuit interactions between specific neuronal subtypes [3,4]. Consistent with this view, new findings in the field of human genetics that has revealed hundreds of gene mutations in the past decade that correlate with such neuropsychiatric disorders; and many of these disease-related genes are linked to synapse formation and function. The expression of these disease-associated genes has recently begun to be systematically mapped to specific brain regions and neuronal cell types [5]. However, it is still not known which properties of these cell classes are affected, and how their functional roles might be altered in dysfunctional brain circuits. Understanding the mechanisms of specific circuit interactions that play a role in animal models of psychiatric disorders can facilitate development of drugs specifically targeting aberrant circuit elements.

### STATE OF THE ART

Currently, functional imaging of neuronal activity in the rodent cortex is achieved using free-space two-photon laser-scanning microscopy [6] together with fluorescent calcium reporters [7]—and this combination provides cellular resolution of activity. Calcium reporters, introduced within the soma, are now widely employed as a robust proxy for electrophysiological measurements. Among such reporters are exogenous synthetic molecules, providing no cellular specificity (e.g. Oregon Green BAPTA-1); or genetically encoded proteins, such as the GCaMP family [8], which can provide cellular specificity through promoter activation and repression [9]. These reporters operate by sensing the intracellular calcium influx following an action potential; this modulates the calcium binding to the reporter and thereby alters its optical cross-section. This stereotypical fluorescent transient is interrogated optically to provide a “report” on calcium influx after the neuron fires.

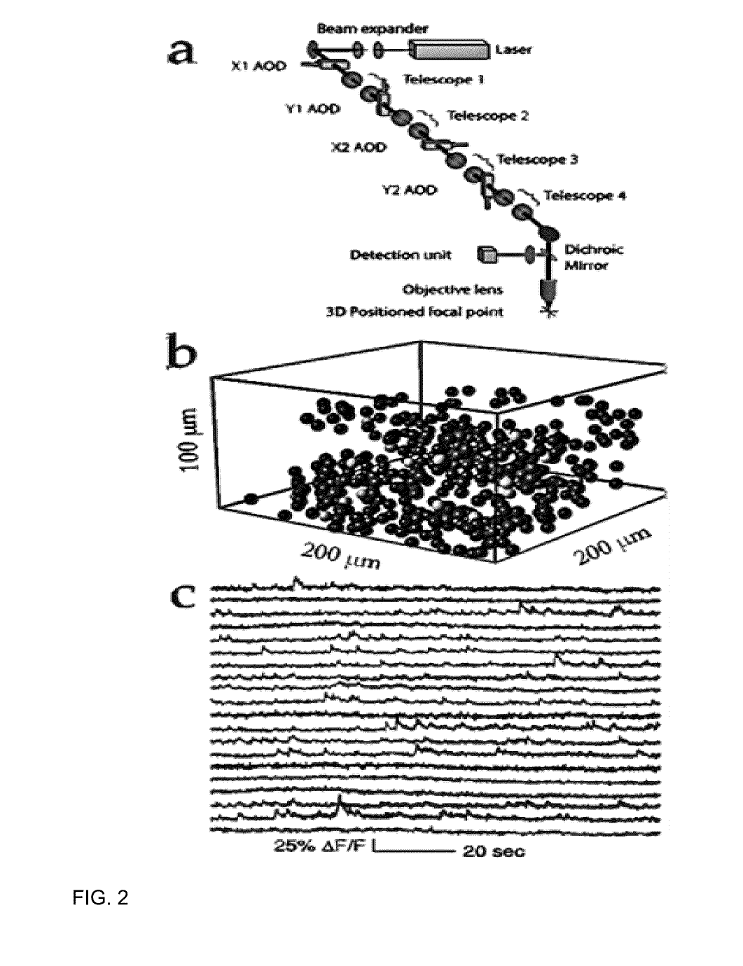

To excite these optical reporters, a serial scanning optical method based on two-photon microscopy is often employed. This involves the simultaneous absorption of two photons by nonlinear processes to induce excitation of the reporter; its subsequent decay to the ground state results in fluorescence emission. Often, near infrared excitation wavelengths are used for biological microscopy; the resulting fluorescence is in the visible spectrum. Because very high photon density is required to induce two-photon absorption, the technique requires a single, tightly spatially- and temporally-focused beam of light generated by pulsed, femtosecond-scale, laser light. Accordingly, to achieve volumetric sampling in three dimensions, a serial, point-scanning methodology or holographic spatial light modulation becomes necessary. In the first methodology the two-photon interrogation voxel, which is typically ˜0.5×0.5×4 m³, is scanned in 3-D, one-location-at-a-time, to map the activity-dependent fluorescence of reporters in individual neurons. Today's state-of-the-art practice utilizes random-access acousto-optic deflectors (AODs), providing ˜10 μs point-access time. Currently, this permits routine mapping of ˜400 neurons in a 3-D volume of 200×200×100 μm³ with the requisite SNR to track spiking activity via the modulated somatic calcium signals (FIG. 2) [10,11,12].

The aforementioned approach has two fundamental limitations that preclude scaling it up to enable functional imaging of large neuronal ensembles spanning extended brain regions: (i) serial optical interrogation, and (ii) signal-to-noise ratio (SNR) degradation with depth.

### Multiplexing Limits of Serial Optical Interrogation

While the aforementioned serial point-scanning optical techniques can provide sub-cellular resolution, they have the significant disadvantage that the total number of scanned voxels is limited, in practice, by scanner speed. This speed limitation also affects current technology for spatial light modulators. This sampling-speed limitation is further exacerbated by the photometric requirement that excitation illumination must dwell at each voxel long enough to achieve requisite SNR. Parallelization of scanned two-photon microscopy in a fixed plane has been demonstrated in brain tissue by using a multiplicity of excitation beams simultaneously followed by conventional wide-field detection. Each beam is encoded with specific binary amplitude modulation to guarantee the unequivocal localization of the fluorescence generated [13]. Such “depth multiplexing”, using four pulsed laser beams with sequential pulses, simultaneously focused at different depths and interrogated with gated detection, has been used to map cortical activity in four optical planes at four different depths [14]. However, it is clear that only a limited number of beams that can be implemented with such a technique; the maximum level of multiplexing that can be achieved is ultimately determined by the laser repetition rate and the reporter fluorescence decay time. To scale this upward requires facing challenging, practical questions concerning the provision of sufficient power in each beam to permit deep imaging in highly scattering neural tissue.

### Signal-to-Noise Ratio (SNR) Limits to the Depth of Optical Imaging

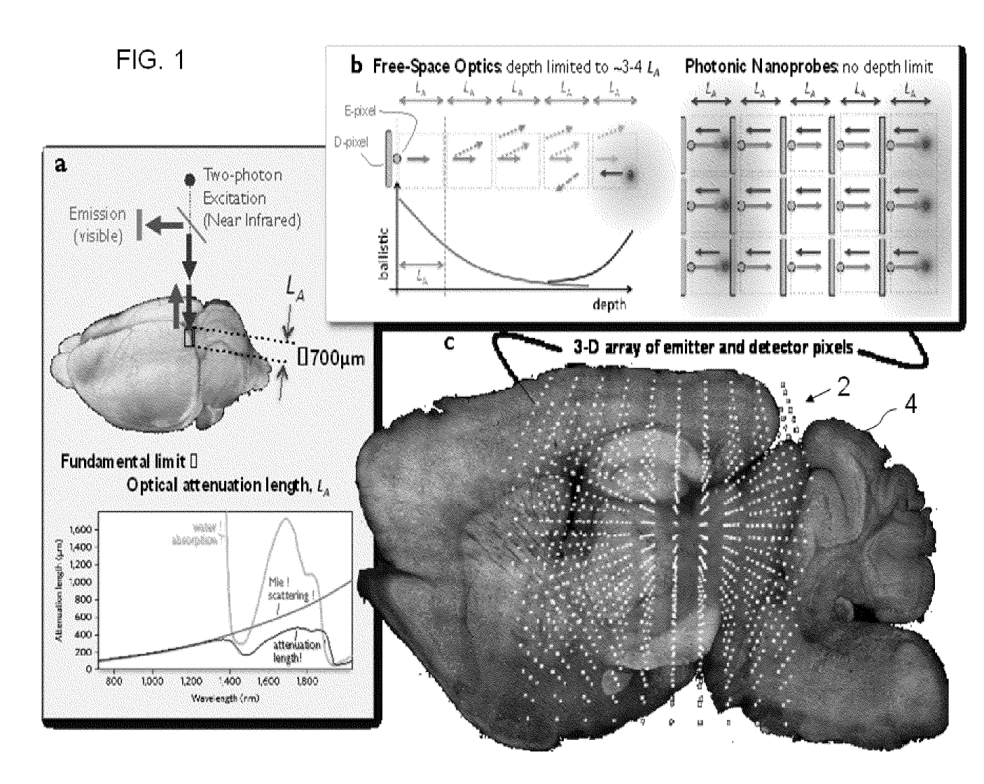

Scattering and absorption limit the ability to deliver ballistic (i.e., unscattered) light with sufficient intensity to achieve tightly focused two-photon excitation deep within the brain. Ultimately, water absorption (FIG. 1a) limits the depth of delivery; in the near infrared (NIR) the maximum attenuation length is L_{A}˜500 μm (FIG. 1a). To overcome this significant limitation, several approaches have been explored. In one, the instantaneous pulse power is increased to enable deeper two-photon excitation, while reducing the pulse repetition-rate to minimize the average power delivered to the tissue. This approach enables recording neuronal activity in populations of L5 neuronal somata up to ˜800 μm deep [15]. However, extending this to achieve even deeper functional imaging becomes very problematic; among issues are generation of out-of-focus fluorescence, even with moderate spatial confinement along the beam, and the onset of nonlinear photodamage in neural tissue.

An alternative approach involves using longer excitation wavelength NIR excitation around 1.6 μm [16]. This is possible by harnessing three-photon absorption processes, but their far smaller cross-sections for existing protein-based reporters imposes as a serious limit on the utility of this approach.

Another proposed approach is to employ adaptive optical corrections to rectify wavefront aberrations that are induced by optical scattering and absorption in brain tissue [17]. In principle, this could restore optical resolution in the two-photon modality, and thereby improve deep-imaging capability. However, the approach is contingent upon measuring, and employing, the precise aberration matrix for a large volume of very heterogeneous media. This is a difficult prospect in highly scattering mammalian brain tissue; further, once obtained it is unclear whether its values would remain sufficiently stationary over typical measurement intervals.

### Emission-Related Limitations

Scattering also acts to dramatically suppress the fluorescence signal accessible via free-space optics. After two-photon excitation, the fluorescent photons originating deep within brain tissue suffer multiple scattering during their propagation. Hence capturing them efficiently after they emerge from the brain's surface requires free-space collection optics with large angular acceptance, i.e. a large field of view, and low magnification [18]. Optics that provide sufficiently large numerical apertures for excitation, and large angular acceptance for light collection, become physically immense. Ultimately, their benefits are limited.

These aforementioned complications in the delivery of excitation light from free space into neural tissue, and the subsequent collection of emitted light after it emerges from tissue back into free-space—to and from regions deep within the brain—have motivated the development of microendoscopy. This method involves implanting a rather large and rigid cannula containing an optical fiber into targeted regions of the brain. After implantation it is then employed for local, functional calcium imaging at the fiber's distal end via one-photon fluorescence excitation of reporters. Although microendoscopy resolves the issue of light delivery and recovery from remote and deep regions of neural tissue, it has very significant limitations. Prominent among these are: i) imaging occurs only within one optical plane near the tip of the endoscope; ii) tissue along the path of the large (typically 0.3-1 mm) implanted cannula/fiber is completely and irreversibly destroyed; and, hence, iii) the approach does not permit studies of vertical structures simultaneously (e.g. cortical layers). Accordingly, the approach is feasible only for acute measurements at the fiber's tip, using direct CCD-imaging [19] or probe-based confocal laser microendoscopy [20]. The goal of integrated neurophotonics is to achieve functional imaging of extended brain regions at arbitrary depths with minimal perturbation of neural tissue. This is not achievable with this method, nor it is compatible while preserving the brain's integrity—given the endoscopic cannula's size.

### Integrated Neurophotonics

Integrated neurophotonics is an entirely new paradigm for functional imaging [21,22,23]. It harnesses recent advances in integrated nanophotonics and functional optical reporters. This new technological approach will surmount the limitations of existing methodologies outlined above. It will enable:

a) Electrophysiological recording and stimulation, with cellular resolution, in highly scattering (mammalian) brain tissue,

b) Access to all regions of the brain, no matter how deep,

c) Complete coverage of all neurons within targeted volumes,

d) Cell-specific interrogation (via protein-based reporters) and complex, finely tuned neurological control (via optogenetics and precisely controlled fields of excitation light),

e) Up-scaling to complex systems that permit simultaneous interrogation and control of millions of neurons, and

f) Mass-production of complete measurement systems using existing microelectronics foundries for ultimate dissemination to the neuroscience and neuromedical communities.

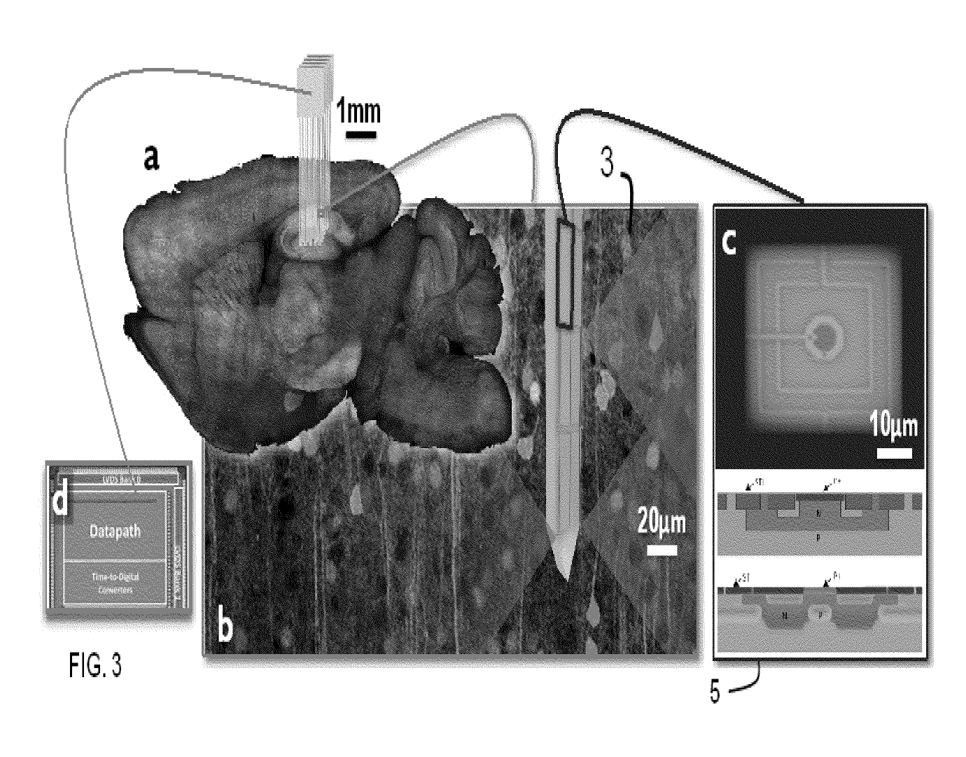

The novel methodology of integrated neurophotonics is based on distributing a dense, 3-D lattice of thousands of emitter pixels (E-pixels) and detector pixels (D-pixels) within the brain on an architecture of neurophotonic probes (FIG. 3). These are configured as long, ultranarrow implantable shanks that provide both acute and chronic functionality. The E-pixel lattice permits local illumination with complex spatiotemporal patterns on the spatial scale of individual neurons; this can be utilized both for local interrogation of optical reporters, and for local stimulation of neurons that have been activated by optogenetic molecules (such as the Opsin family). The D-pixel lattice provides a multiplicity of distributed and spatially discrete points-of-collection. Its data (which, from each detector in the array, may be realized in the form of intensity time records, actual photon counts, or other embodiments), in turn, enables both spatial mapping of individual neurons in this locale by triangulation, and monitoring the cell-specific activity of the reporter-labeled neurons by optical spike sorting. As illustrated in FIG. 1b, through proximal illumination and detection, the novel architecture enabled by integrated neurophotonics bypasses the conventional limits of light dispersion and attenuation in brain tissue. Specifically, through detection that is both massively parallel and distributed, this methodology circumvents limitations to multiplexing that arise with existing, free-space optical methods—including the most advanced serial point-scanning and spatial light modulation methods. Importantly, it enables imaging at any depth in the brain; the shanks on which the E- and D-pixel arrays are embedded can be engineered to be as long as necessary. An E- and D-pixel array 2 inserted into brain tissue 4 is shown in FIG. 1C. FIG. 3 shows a cell body 3 of an individual neuron, and cross-sections 5 of the layer structure of an SPAD.

In the some embodiments, tracking of somatic calcium transients that arise from neural spiking can be read out, in parallel from a multiplicity of labeled neurons, to acquire the ensemble of their individual fluorescence time-series. This information can be retrieved in the time domain by gated integration of illumination or by nanosecond optical interrogation and time-correlated photon counting. This is enabled by the arrays of integrated-nanophotonics-based emitters (E-pixels) that operate in concert with detector arrays (D-pixels). Among possible embodiments for the individual D-pixels are gated CMOS photodetectors or single-photon avalanche photodiodes (SPAD). The D- and E-pixel elements can be specially designed for embedding as large arrays onto ultranarrow shanks for acutely or chronically implanting into neural tissue. The data acquired from such an integrated neurophotonic system can yield fluorescence time records for the entire ensemble of active neurons within the volume probed. New protocols, which are termed “optical spike sorting”, enable such data extraction from the raw data provided by the D-pixel arrays. These protocols employ model-based clustering algorithms, similar to spike sorting protocols used in multi-site electrical recording.

### Engineering of Neurophotonic Probe Arrays

### Integrated Neurophotonics

The integrated neurophotonics technology images neuronal activity from inside the brain by distributing thousands of local light sources and detectors within large volumes arbitrarily deep in the brain. This can be achieved by distributing light emitters and detectors throughout the brain on, for example, ultra fine silicon shanks (on the order of ˜25 μm wide and 15 μm thick), and then controlling them with integrated nanophotonic and nanoelectronic chips (FIG. 3). In the concrete example described herein, one such module can enable mapping the activity of all neurons within a 1 mm³ volume of brain tissue (˜100,000 neurons in the mouse cortex). Further, the modular architecture of these neurophotonic probe arrays can enable tiling them to cover extended neural circuits, meanwhile maintaining the capability of dense recording. For example, recording simultaneously from one million neurons throughout mouse visual cortex will be possible.

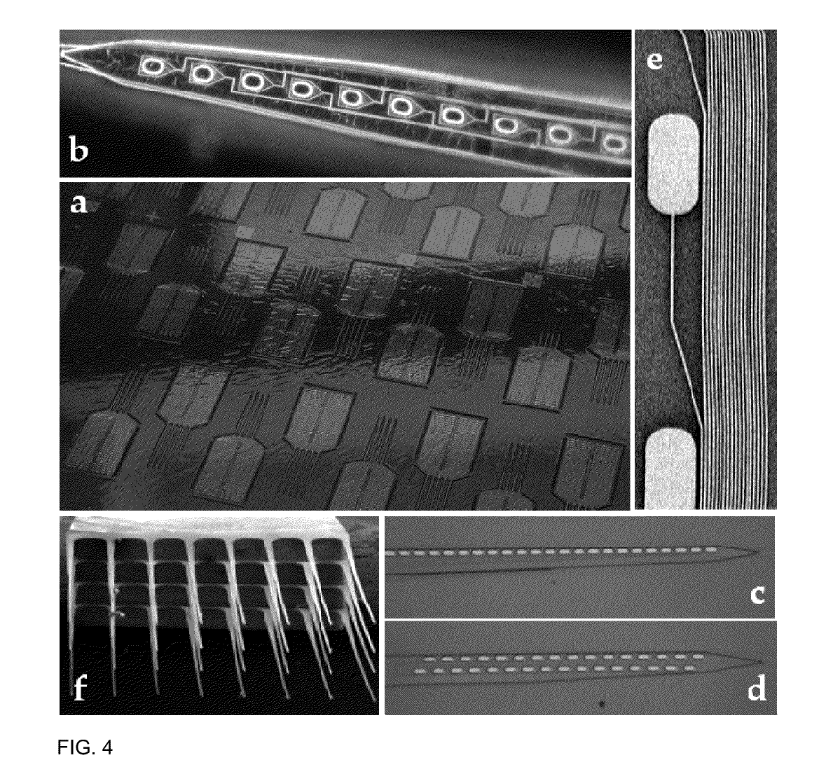

The integrated neurophotonic systems described herein merge three key technologies. First, they leverage current developments in fabrication of advanced silicon-based nanoprobe arrays for deep and massively multiplexed electrophysiological recording in brain tissue (FIG. 4). Integrated neurophotonics, in effect, substitutes the electrical components comprised within the electrophysiological probes with integrated optical elements. These integrated optical elements are achieved through state-of-the-art, chip-based nanophotonic technology. The requisite core technologies are already being realized at the wafer scale within photonics foundries (mass photonics-chip production facilities). Thus, the approach of integrated neurophotonics can provide instrumentation that is readily capable of mass production and is practical enough to permit, ultimately, its wide deployment.

### Overview of a Prototype System Architecture

To provide a specific embodiment of one possible embodiment, an integrated neurophotonic system is envisioned that coalesces N_{E}=2050 E-pixels and N_{D}=2050 D-pixels within a volume of 1×1×1 mm³ of brain tissue, using an array of 25 ultra fine shanks. The detection pixels are realized as photon-counting detectors, specifically, as single-photon avalanche photodiodes. The stimulation pixels are realized as E-pixels located at the termini of integrated nanophotonic waveguides running along the probes shanks; these termini are spatially distributed along the shank in configurations determined by experiments and computations as providing the most ideal raw data for “de-mixing”. At the top of the integrated photonics probe, these waveguides efficiently interface with the sources via a separate, active photonics-source chip at the layer head.

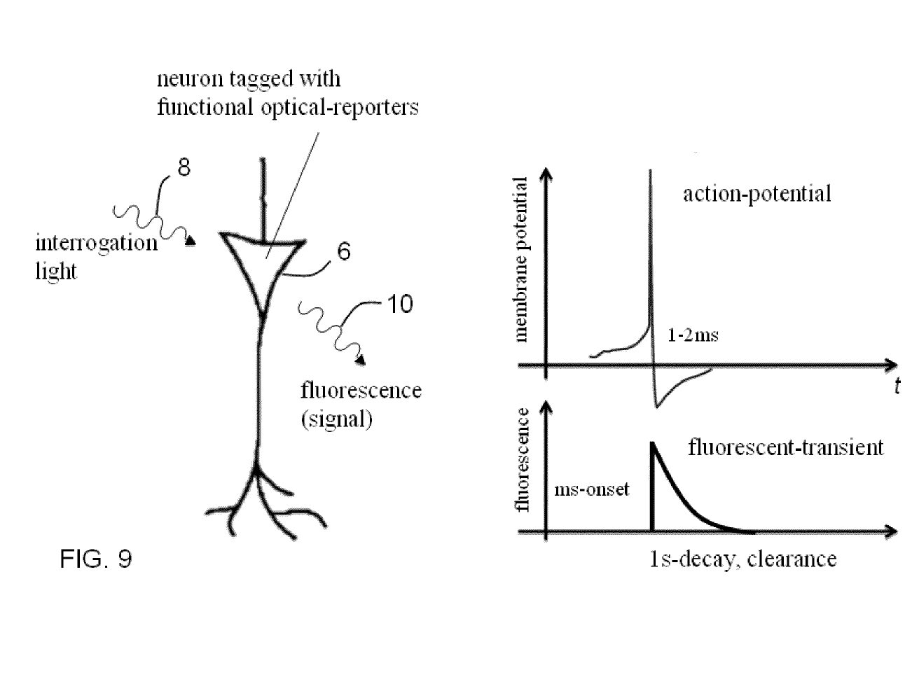

Referring to FIG. 9, a neuron within neural tissue that is prepared with functional optical reporters is shown. This neuron 6, tagged with a functional optical reporter, is probed using an interrogation light 8, the result of which is emitted fluorescence (signal) 10. Each time a neuron fires an action potential, which occurs (very approximately) on millisecond timescales, the fluorescence signal is transiently modulated

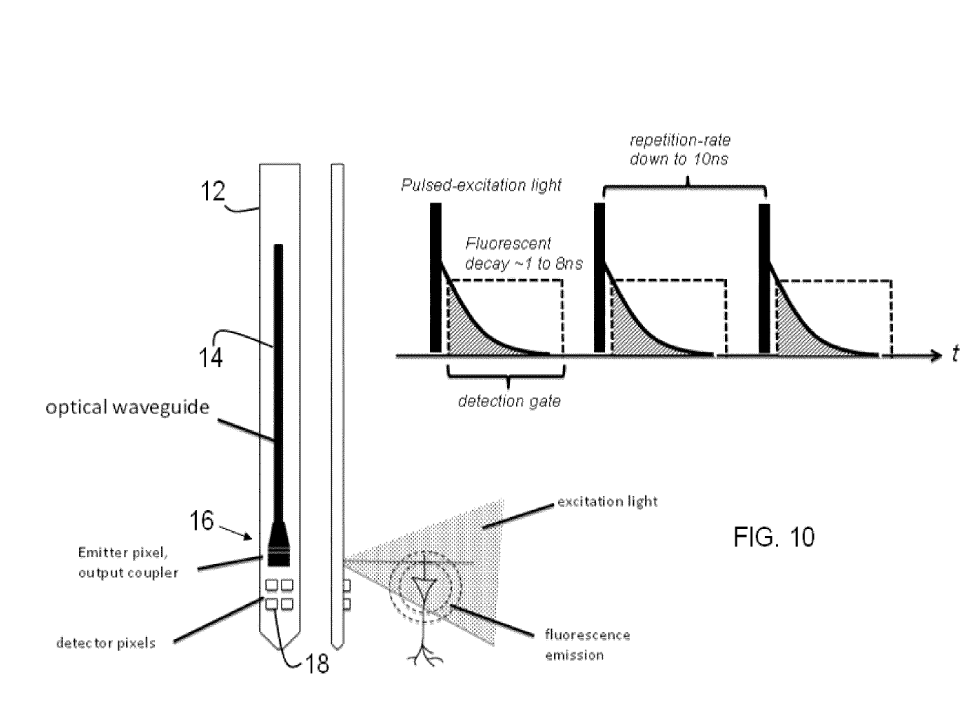

FIG. 10 shows an embodiment of co-integration of emitter and detector pixels on the same shank to illustrate the process by which neuronal activity is optically probed using a fluorescence activity reporter. The left panel of the figure shows front and side views of a single integrated neurophotonics probe shank 12. The front view shows an optical waveguide 14, an emitter pixel 16, an output coupler, and four detector pixels 18. The side view of the shank illustrates excitation light being emitted from the emitter pixel. In this illustration, a single optically-labeled neuron lies in the path of the excitation light, resulting in fluorescence emission by the ensemble of optical reporters expressed within the neuron. The right panel of the figure shows the time evolution of the pulsatory excitation light and the induced fluorescence. This light output from the optical reporter decays over a time interval of approximately 1 to 8 ns (shaded area under curve), and can be probed with a repetition rate of as little as 10 ns. The dotted line rectangle represents the detection gate, the interval over which the detectors are active and collecting emitted photons from the labeled neuron. By staggering the excitation and detection time windows, direct detection of the excitation photons is circumvented.

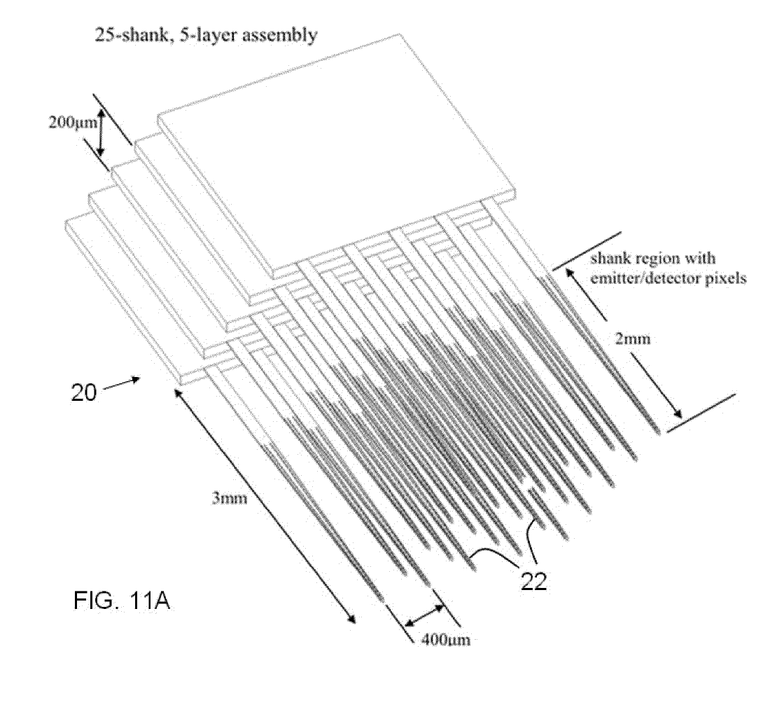

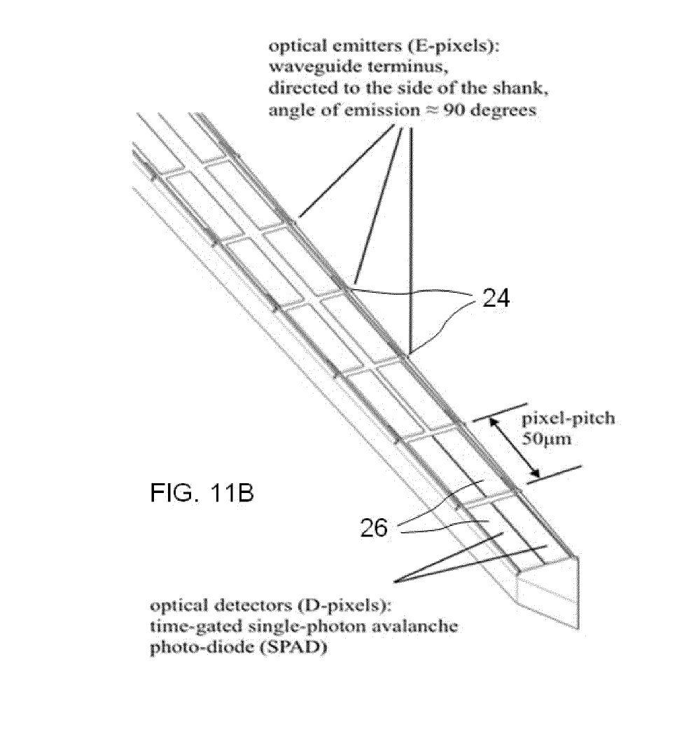

Referring to FIGS. 11A and 11B, an embodiment of the multi-layer assembly of a probe ensemble is shown. FIG. 11A shows an embodiment of such a multi-layer assembly, illustrating a probe 20 with 25 shanks in a 5-layer assembly with five shanks per layer. In this embodiment, there is a distance of 200 micrometers between each layer. Each shank 22 has a length of 3 mm. In this embodiment, the “active” region of the shank with the emitter and detector pixels (shaded rectangles) is located within the distal 2 mm regions on each shank. In this embodiment, there is a distance of 400 micrometers between shanks. FIG. 11B illustrates the terminal end of a single shank, depicting optical emitters (E-pixels) and optical detectors (D-pixels), repeated with a pixel-pitch of 50 micrometers. The optical emitters 24 (E-pixels) in this embodiment consist of a simple (blunt) waveguide terminus, and at the termini are directed to the side of the shanks. The angle spread of emitted light from each E-pixel is approximately 90 degrees. The optical detector elements (D-pixels) in this array, for this embodiment, are optical detectors 26 based on time-gated single-photo avalanche photodiodes (SPADs).

Fabrication of photonic probes involves a variety of standard lithographically-based micro- and nano-fabrication steps, concatenated in a unique sequence to realize this novel technology.

The shanks can be patterned from layered materials, such as silicon-on-insulator (SOI) substrates, using techniques of surface micromachining. The topmost silicon layer of the SOI substrate in this instantiation provides the structural material for the probe shanks themselves. Alternatively, bulk micromachining techniques common to microelectromechanical systems (MEMS) processing can be employed to realize probe shanks through processes such as selective chemical etching.

Integrated photonic waveguide elements are fabricated using standard methods and in standard requisite geometries by selectively patterning layered materials possessing different indices of refractions. For visible wavelength instantiations, for example, silicon nitride and silicon oxide layer can provide the requisite light confinement.

Emitter elements are fabricated at the termini of each integrated photonic waveguide on the probe chip. These may be as simple as a blunt end of a waveguide, lithographically etched to provide a surface perpendicular to the axis of the waveguide. More advanced E-pixel termini—as mentioned, involving conventional lens elements, planar lens elements, or metamaterials-based elements—can be employed to engineer the spatial profile of the light beam emerging from each terminus.

Detector elements are described below. These can take the form of integrating elements providing grey scale values for the illumination collected at each D-pixel, or photon-counting elements that quantify the precise numbers of photon each D-pixel collects during a specified time interval.

A multi-channel optical interface between the head of the integrated neurophotonics probe and the separate, active photonics-source chip is present in the embodiment. The coupling elements used to form such an interface—which are present on both the integrated neurophotonics probe and the separate, active photonics-source chip—shall be termed chip-to-chip coupler arrays. A variety of methodologies for providing such coupler arrays are possible. For example, opposing arrays of planar grating couplers can be used on both chips and then registered spatially as the chips are brought to proximal face-to-face alignment. Efficient transfer of photonic signals is then mediated in the free-space region across the gap between the aligned chips. Importantly, such a stand-off between the integrated neurophotonics probe and the separate, active photonics chip can provide thermal isolation from system elements operating with high power dissipation—this can very effectively circumvent very critical issues of heating in delicate neural tissues.

The aforementioned grating-coupler-mediated chip-to-chip photonic interfaces can be augmented or supplanted by faceted mirrors, which may be fabricated by various standard lithographic and etching methods. These can be further augmented, or supplanted, by lens elements that can provide enhanced efficiency. Among possible lens elements are conventional structures comprising three-dimensional shapes patterned from materials with different indices of refraction, or those comprised of planar lithographically-defined focusing elements (such of zone plates, etc.), or alternatively those comprised of metamaterials-based elements.

Arrays of miniature light sources are employed to drive the E-pixel arrays. These photonics-source arrays, which are located on the separate, active photonic-source chip, deliver light through the chip-to-chip coupler array to the waveguide arrays that run along the integrated photonics probe and ultimately terminate at the array of E-pixel elements.

Two instantiations exemplify distinct realizations of such photonics-source arrays.

First, a single source of fast, pulsatory illumination may be employed. Among possible single-source elements can be pulsed laser sources, supercontinuum laser sources, etc. In this instantiation the single incoming light beam is divided up in the N daughter beams by an optical power splitter, then each daughter beam is modulated by integrated photonic elements to temporally encode the requisite time profile that is ultimately desired to realize specific patterns of illumination emerging from the E-pixel array. This is further described below.

Second, an array of microscale light sources can be employed. (Several possible instantiations are described below.) Each photonics-source element in these arrays provide excitation light at the requisite wavelength(s) for the optical reporters or optical effectors, and also permit fast modulation of the light to permit formation of complex illumination—patterned both spatiotemporally, and also spectrally (if desired)—from the E-pixel arrays. Given that typical florescence lifetimes can be of nanosecond duration, modulation rates exceeding 1 GHz are desirable.

To drive the E-pixels, optical sources are required. For pulsatory or time-gated implementations, these must produce light pulses with shorter temporal duration than the lifetime of the optical reporters (for recording local activity) or effectors (for stimulating local activity) that are employed. Typical fluorescent lifetimes are in the several nanosecond range, hence sub-nanosecond pulses at the desired wavelength will be required. Many measurements are desired during the time course of a typical activity event. For example, there will be a time window of up to tens of milliseconds during which detection of neuronal action potentials via intracellular Calcium reporters is possible. In this case, measurement repetition rates on the order of one hundred MHz will provide optimal sampling of fluorescence excitation without saturation. Such measurements can be implemented in two possible ways: (i) A free-space mode-locked laser can be coupled to an integrated optical splitter on the photonic chip which will produce NE=2050 individual light sources. Each optical channel of the splitter comprises an on/off switch (using all-optical, acousto-optical or electro-mechanical modulators) for creating a vast multiplicity of illumination patterns from the emitter pixel array. The 2050-optical channels are carried by waveguides running on the fine shanks, which terminate at desired locations along the shanks. At these termini, various optical elements can be employed to control the angular profile of the termini-emitted light. (ii) In a second instantiation, NE=2050 individual integrated light sources (laser diodes, LED, resonant-LED) can be coupled—for example, via grating couplers, mirrors, planar, nanostructured, or metamaterial-based optical elements, and other implementations—to integrated optical-modulators or optical gates in order to generate sub-nanosecond light-pulses with rates on the order of 100 MHz and sub-nanosecond duration. The patterning of light emission from the E-pixel array termini can be achieved by on/off or continuous modulation of the individual sources driving each E-pixel element; this can be achieved either by direct electrical control of the sources, or by a downstream array of optical modulators. In the latter case, the light modulator outputs would couple to on-chip waveguides running along the fine shanks to the aforementioned termini (with any included optical elements to control the spatial profile of emission).

For detector pixel realizations based on single-photon detectors (such as SPADs), signal output is in the form of electrical pulses corresponding to the detection of individual fluorescence photons. These pulses, characterized by their duration and amplitude, can be be sent through fast electrical lines, e.g. coplanar waveguides, that run along the fine shanks up to a time-to-digital-converter circuit located on the probe head. This readout circuit then generates a digital quantification of the fluorescence photon counts detected by each pixel during the relevant integration time dictated by to the reporter kinetic. Electrical commands, i.e. time-gates and bias, to the detector pixels will be provided by the same circuit comprising the time-to-digital circuit, and can be routed to the detector pixels by electrical connections running on shanks as well. The readout detector circuitry subsequently outputs digital signals to computer data acquisition interface.

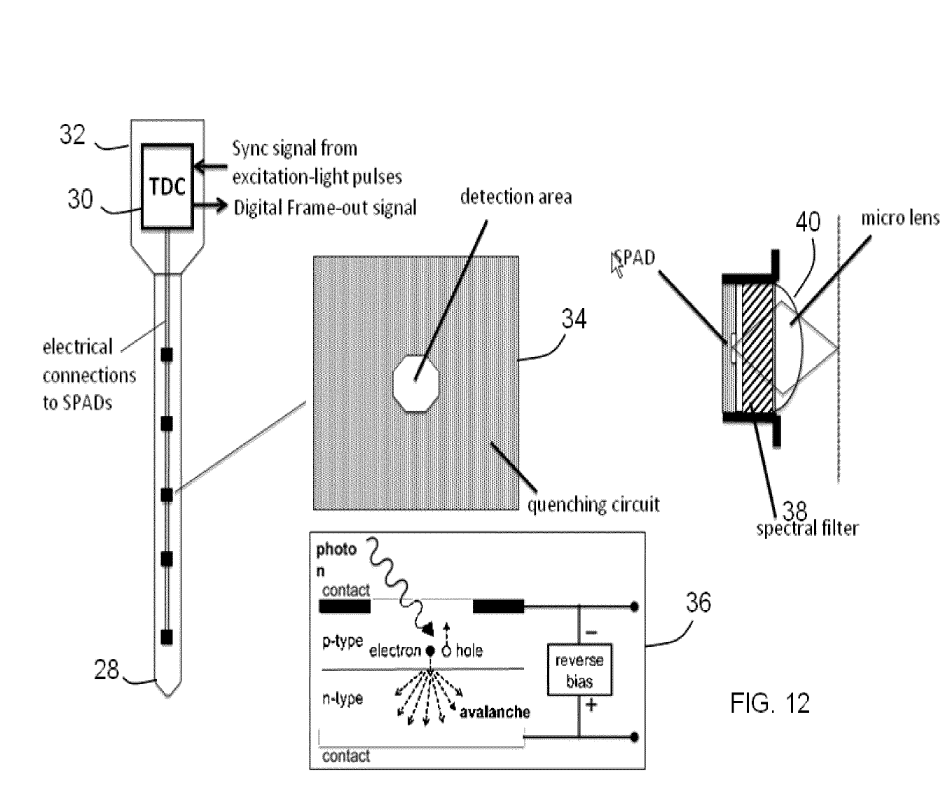

Referring to FIG. 12, the photon-counting mode of operation for a single-photon avalanche photodiode (SPAD) is shown, which is embedded upon an integrated neurophotonics probe shank. The left side of the figure shows an embodiment of a single shank 28 with 5 SPAD detector pixels (represented by filled black squares on shank). In this embodiment, the SPAD detector pixels are connected electrically to time-to-digital converter circuitry 30 (TDC, bold-line rectangle) on the head 32 of the probe shank. The excitation-light pulses provide a clocking signal (sync) for the TDC (bold-line arrow into TDC), and digital frame-out (bold-line arrow out of TDC) signifying the end of a given data acquisition interval are also illustrated. The top portion of the center panel of the figure shows an enlarged view of a single SPAD detector pixel 34. In this example, the active detection area (represented by octagon) in shown in the center of a quenching circuit (represented by large shaded square). The lower portion 36 of the center panel of the figure illustrates a single photon (wavy arrow) interacting with the sensing area of the SPAD detector pixel, resulting in an avalanche of electrons. The right side of the figure illustrates a side view of a single SPAD detector pixel. A spectral filter 38 (diagonal striped rectangle) and a micro-lens 40 are also shown.

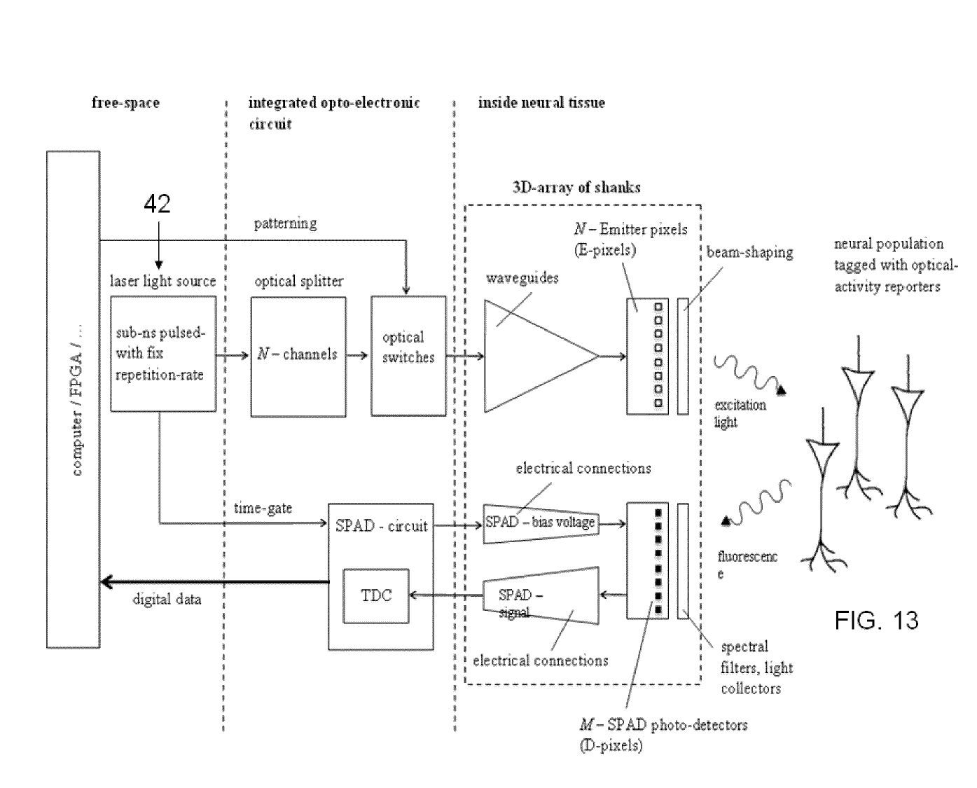

FIG. 13 is a block diagram for the full architecture of one embodiment of an integrated neurophotonics data acquisition system. Principal elements of this integrated system are illustrated. In the diagram, a computer (FPGA, etc.) and laser light source 42, both located in free-space, are connected to the integrated optoelectronic circuit, allowing for control of the optical signal (patterning, optical splitting, etc.) that is subsequently delivered to the waveguides on the shanks inside the neural tissue. These optoelectronic elements also provide time-gated control of the SPAD-array circuitry. Each waveguide, located upon the 3D-array of shanks implanted into neural tissue, terminate at an emitter pixel (E-pixel). These E-pixel elements provide beam-shaping of the emitted light as required. In this illustration, excitation light emitted from E-pixels is depicted as interacting with a population of neurons labeled with optical reporters; upon excitation they emit fluorescence. This fluorescence signal can be filtered to suppress spurious background fluorescence (for example, using spectral filters, light collectors, etc.) and then detected by detector pixels (D-pixels) which are also located on the 3D-array of shanks implanted within the neural tissue. In this example, the D-pixels are an array comprised of a number (M) of SPAD photo-detectors, which, via electrical connections, receive input (bias-voltage from the SPAD circuit) and also provide signal output (which represents the photon count) to the time-to-digital converter (TDC). The TDC, in turn, provides digital data back to the computer controller (FPGA, etc.) that is directly correlated to the photon count detected by the implanted D-pixel.

The shank-array spacing and the pixel pitch together delineate a unit volume that is interrogated by adjacent nanophotonic emitter/detector pixels (FIG. 3). For the prototype example described here, this unit volume is 4.0×10⁻³ mm³; for a typical cortical density of ˜100,000 neurons/mm³ this comprises ˜400 neurons. Functional reporters can be employed that absorb at k=480 nm when bound to Ca²⁺. The prototype architecture can position at least 18 detector and 18 emitter pixels within one optical attenuation length, LA, the average distance a photon traverses between scattering or absorption events.

The optical attenuation length of the neural tissue at the wavelength of interest can be experimentally evaluated by measuring the loss of optical power from a plane-wave like illumination going trough a tissue slice of known thickness.

At 480 nm the optical attenuation length is typically deduced to be on the order of 200 μm [6]. Together, the emitter pixel array makes possible illumination of each unit volume's contents with at least to 2¹⁸˜262,000 different patterns. For each illuminated unit volume, readout of the induced fluorescence will be possible from 18 independent positions for each of the chosen illumination patterns. Together, this provides >4 million combinations of measurements for every unit volume. The >1 B combinations available with the 25-shank module yield sufficiently dense coverage of all ˜100,000 neurons within the 1 mm³ volume of brain tissue to permit their unique and individual sampling.

The total optical power of one emitter pixel can be in the range of ˜10 to ˜100 μW at visible wavelengths (λE=480±10 nm). Output powers in this range and below preclude induction of tissue damage near the E-pixel termini. Excitation and collection pixels can be oriented orthogonally on the shanks to minimize direct spectral feedthrough, although the most effective method for suppression of direct E- to D-pixel optical coupling (feedthrough) is through use of time gating. This enables staggering the excitation and detection time windows; during the latter the illumination can be completely turned off allowing fluorescence from optical reporters to be sensed with minimal background. The requisite readout bandwidth can be dictated, at the low end, by reporter kinetics. This is in the range of ˜100 Hz when using slow calcium reporters such as genetically encoded GCaMP proteins or exogenous BAPTA-based molecules like Oregon-Green®488BAPTA-1 (Life-Technologies). At the high end, bandwidths of order 1 kHz will become achievable with use of fast voltage sensitive reporters such as genetically encoded Arch (Archaerhodopsin)-based fluorescent voltage sensor or dyes like ANEP (aminonaphthylethenylpyridinium) and variants. This will enable implementation of rapid changes in patterned illumination to realize optimal spike sorting protocols. A total detector-integration time of 10 ms per readout is envisioned. This is compatible with the targeted mean irradiance of 1016 photon/s/cm2 within the unit volume, and with the kinetics of the current GCaMP reporters.

### Photon Counting D-Pixels

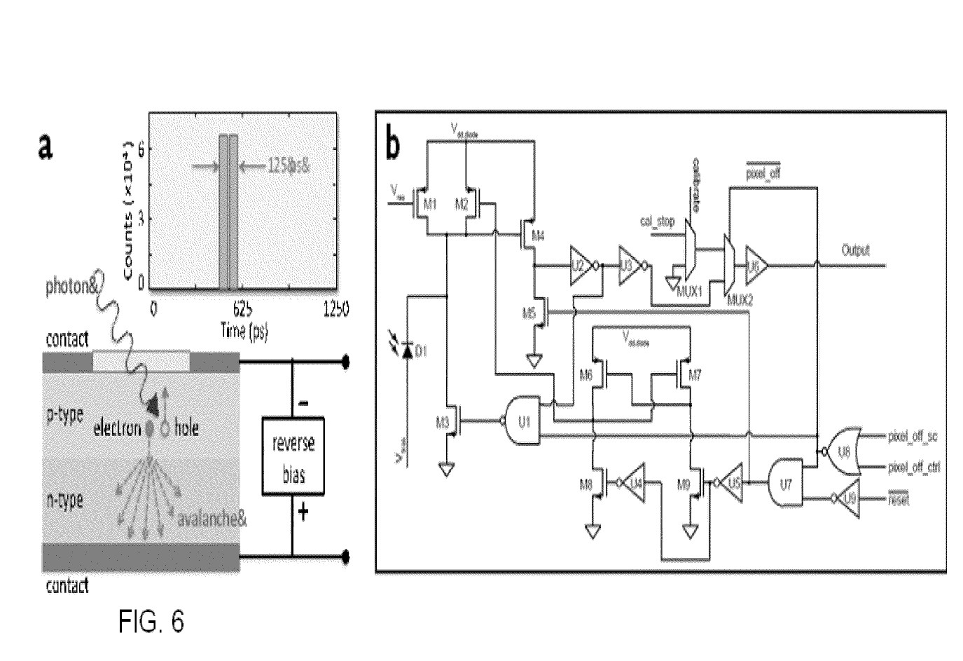

CMOS-compatible sensors integrated on the shank could take two forms. In traditional CMOS image sensors (such as those employed in the cameras of many light microscopes), photocurrent is integrated onto the reverse-biased photodiode on which it is generated, producing a voltage signal that is directly proportional to the light intensity. The sensor itself is “low-gain”; that is, it produces fewer electrons that incident photons. An alternative sensor is a “high-gain” one that produces many electrons from a single incident photon. The photomultiplier tube is an example of such a photon-counting sensor. In the solid-state world, detectors providing single-photon sensitivity take the form of single-photon avalanche diodes (SPADs), which are photodiodes biased beyond their avalanche breakdown voltage. When a photon is incident, it creates an electron-hole pair with a probability known as the photon detection probability (PDP), which triggers carrier avalanche within the diode (FIG. 6a). Upon avalanching, external circuitry reduces the voltage across the diode below the avalanche voltage, quenching current flow (FIG. 6b). The voltage is then raised again to await the arrival of another detected photon. Very recently, several groups, including the inventors′, have successfully built high-performance SPADs using conventional CMOS processes [24-32]. Noise in SPADs is manifested as dark count rates [33] or after-pulsing [34].

These high-gain single-photon detectors allow one to accurately measure the arrival time of individual incoming photons, in a measurement technique known as time-correlated single-photon counting (TCSPC). For each SPAD, this requires a time-to-digital converter (TDC), which accurately captures the arrival time of the photon in digital form. The combination of a SPAD and pulsed excitation light, to which one can synchronize the measurement, one can easily time-gate the fluorescence measurement and eliminate the feedthrough of interrogation light to the detector.

### Single-Photon Avalanche Photodiodes (SPADs) and Pixel-Level Control Circuitry

SPADs are used in a time-correlated single-photon counting mode in which arrival time histograms are recorded through time-to-digital conversion of photon-activated pulses from the detectors. SPAD detection limits are determined by noise in the form of the device's dark count rate (DCR). DCR is dominated by avalanche events that are triggered by the thermal generation of carriers from recombination-generation (RG) centers within a diffusion length of the multiplication region of the SPAD. The shallow trench isolation (STI) that is used to separate devices in modern CMOS processes creates a relatively defect-rich interface and a significant source of RG traps. The inventors have figured out approaches to eliminate these STI interfaces from the multiplication region of the SPAD structure, allowing the formation of SPADs in a rather advanced 0.13-μm CMOS node[30]. These SPADs have an octagonal photosensitive area with a diagonal extent of ˜5 μm (FIG. 2c). The measured reverse bias breakdown voltage (V_{br}) is −12.13 V. For photon counting, the diode is operated in Geiger mode, biased beyond V_{br} by an overvoltage (Vₒᵥ) but drawing no current until a free carrier in the multiplication region triggers an avalanche. This mode of operation requires a quenching circuit, the simplest form of which is a resistor in series with the diode. When an avalanche is triggered, a current flows through the resistor causing a voltage drop, which leads to the voltage across the diode rising above V_{br}, halting the current; the associated RC time constant to return to a reverse bias of (V_{br}−Vₒᵥ) defines the deadtime for the SPAD.

In existing devices [30] relatively shallow junction depths (300 nm), which result from using the p+ mask for a PFET source and drain implant, cause the photon detection probability (PDP) to peak at ˜425 nm. Pixel implants can be optimized (using lighter doping) to achieve higher PDP at longer wavelengths.

To reduce deadtime, active quenching circuitry is often utilized. In the inventors' previous SPAD design [31,32], the pixel circuitry shown in FIG. 3b was employed. In order to quench the device, the voltage across the SPAD must be reduced to below its breakdown voltage, V_{br}. In this design, a PFET device M1 is used as the quenching resistor. A tunable voltage, Vᵣₑₛ is applied to the gate of M1, which allows the drain-to-source resistance, R_{ds}, of the device to be adjusted. After the SPAD has been quenched, it must be reset before it can be used to detect another event. In an active quenching approach, the wide-channel PFET device, M2, performs reset. In addition, the NFET, M3, is used to hold the bias across the SPAD below breakdown and can be used to prevent the SPAD from resetting or to disable the SPAD completely. The pixel circuitry contains several additional circuits for diagnostics and to allow the pixel to be controllably disabled. This particular pixel design in a 0.13-μm CMOS process gives a 48-μm pitch but a fill factor of ˜1%. For an on-shank implementation, the pixel circuitry can be greatly simplified to allow this pitch to be reduced to less than 30 μm. Additional development work can increase the photodiode active area by a factor of two without a significant increase in dark count rate, allowing a 4× improvement in the fill factor. Introduction of laser-patterned dielectric microlens technology can further improve the effective fill factor to greater than 80%. This technology is already in active use in CMOS imagers and all major microphotonics foundries support their fabrication.

### Time-to-Digital Converter (TDC) Circuitry

A time-to-digital converter (TDC) is used to measure the arrival time of the first photon detected during each measurement window. In previous designs [31,32], the design targets for this TDC were for a 62.5 ps resolution with a 64 ns range (FIG. 6a). In prior work, the TDC is based upon delay-locked loop (DLL) architecture with a synchronous counter. This was chosen because of its well-defined precision and dynamic range, its fast conversion speed, and the ease with which it can be shared among many pixels in the array.

If a 10-bit time value is output for every pixel after each excitation event, with a laser pulse rate of 20 MHz, an off-chip data rate of 1.8 Tbps would be required for the array. This data rate, however, does not reflect the sparseness of the data. In particular, TCSPC experiments typically record a photon hit for only 1-2% of laser repetitions. Through the use of an event-driven readout approach, sparseness is exploited in our design to reduce the average data rate to approximately 18 Gbps. To achieve this, the time data for each pixel are appended with a valid bit that indicates whether a pixel event has occurred. This valid bit is used to control the flow of data out of the array such that only data associated with pixel events are allowed to pass.

Current power dissipation is about 80 μW for the SPAD and 30 μW for each TDC channel. For a total power budget of 5 mW down each shank, which creates less than 0.5 degree of heating at the tissue, about 40 SPAD detectors per shank can be supported. The D-pixel arrays can be increased if power consumption is further reduced. The current data path design consumes in excess of 4 mW/channel through inefficient use of the data sparseness, consuming a significant amount of power “clocking zeros” through the design. This can be expected to be reduced to less than 500 μW/channel in a new design

### Optical Spike Sorting

The data acquired from an integrated neurophotonics functional imaging system will be inherently complex; algorithms for “de-mixing”—that is, transforming the acquired photon counts at each D-pixel in the detector array into time records of activity from individual neurons—will need to be developed. Each E-pixel illuminates a local volume within the brain, which contains multiple neurons and the surrounding neuropil. The resulting fluorescent emission is then measured from many perspectives, via multiple detectors within roughly one or two attenuation lengths, L_{A}, from the emitting neuron. The overarching goal of the prototype described here is to enable recording all of the activity of a 1 mm³ volume of the mouse cortex, containing ˜100,000 neurons, with single cell resolution. This is feasible using one 25-shank array of photonic probes. Using 2050 E-pixels that are separately activatable to create complex patterns of local illumination, and simultaneously monitoring the evoked fluorescence with 2050 D-pixels, provides access to a space of almost 5 million measurement configurations. More complex patterns of illumination, beyond simple on/off modulation, can further increase the richness, i.e. the complexity, of this measurement space. Reconstructing the sources of activity, i.e. identifying the activity of the neurons in this volume, which shall be termed “optical spike sorting” is an important signal decomposition problem, and development of efficient algorithms for this can be produced.

Specific design considerations help to make this problem more tractable. Initial use of the GCaMP6 activity reporter is envisioned; this reporter has different optical properties that depend upon whether it is in the calcium-bound or—unbound state—the former corresponds to conditions of high local Calcium concentration. Conversely, this reporter has a very low fluorescence level under conditions of low calcium concentration. Accordingly, the amount of background noise from the inactive neurons within an illuminated volume can be very low. Second, by placing detectors and emitters much closer to the neurons than in traditional microscopy, the efficiency of illumination and detection increases dramatically, improving the signal amplitude. Third, because of the local configuration of the illumination, a single emitter excites only a small subset of the unit volume. This optical “sectioning” further facilitates decomposing the datasets into individual neurons. Specifically, a particular configuration of activated E-pixels can only excite a subset of the neurons that are active (i.e. “spiking”), and a particular configuration of D-pixels will only collect from another subset of the neurons. This reduces the entire decomposition problem into a number of overlapping smaller problems.

The signal decomposition problem can be formulated as follows:

$$d ( t ) = n ( t ) + {\sum }_{i = 0 , ⋯ , N} {\sum }_{τ = 0 , ⋯ , T} {r}_{i} ( τ ) {δ}_{i} ( t )$$

where d(t) are the observations over time, measured from the detectors with each combination, i.e. “pattern”, of excitation. Note that the d(t) are samples in ˜5 million dimensional space for the prototype example (i.e. 2050 D-pixels and 2050 E-pixels), n(t) is the noise (which may be partially correlated across channels), δₜ(t) is 1 when neuron fires an action potential and zero otherwise, and rᵢ(τ) is the kernel that describes the fluorescence activity. The kernel can be largely predicted based on the geometry between a neuron and the emitter and detector arrays—as well as the known, typical time course of the reporter's fluorescence signal (however, it will likely benefit from some degree of fitting). Finding the latent variables δᵢ(t) allows solving for the set of spike times that would mostly likely result in the given dataset. Latent variable problems in neuroscience such as this are typically solved using probabilistic techniques such as particle filters [35]. However these methods can be optimized to deal efficiently with the high-dimensional data space and the multiplicity of neuronal sources involved in the present approach. Alternative methodologies such as detecting events, clustering them, and “unpeeling” them to recover the underlying activity hold additional promise for such analyses [36].

Although the system has been described mainly in connection with neural tissue, the methods, systems, devices and other embodiments are also applicable to other tissues, such as muscle. Examples of cells in that can be investigated and appropriately labeled include, but are not limited to, neurons, glial cells and muscle cells. The tissue can be in an organism, or can be explanted tissue.

In some embodiments, the tissue can be prepared by optogenetic methods. In optogenetics, photoactivatable proteins, receptors or channels can be incorporated into tissues, making the tissues photo-responsive (Yizhar, O., et al., Optogenetics in Neural Systems, Neuron 71, 2011; Zhang, F., et al., Channelrhodopsin-2 and optical control of excitable cells, Nature Methods 3(10), 2006; Boyden, E., et al., Millisecond-timescale, genetically targeted optical control of neural activity, Nat. Neurosci 8(9), 2005).

Although various components of the probe device have been described separately, it should be understood that any embodiment of one component is contemplated to be combined with any embodiment of another component. Thus, for example, any combination of optical emitters, optical detectors, fiber shanks and optical sources is envisioned. Similarly, although various features of the methods have been described separately, it should be understood that any embodiment of one feature is contemplated to be combined with any embodiment of another feature.

The present invention may be better understood by referring to the accompanying examples, which are intended for illustration purposes only and should not in any sense be construed as limiting the scope of the invention.

### Example 1

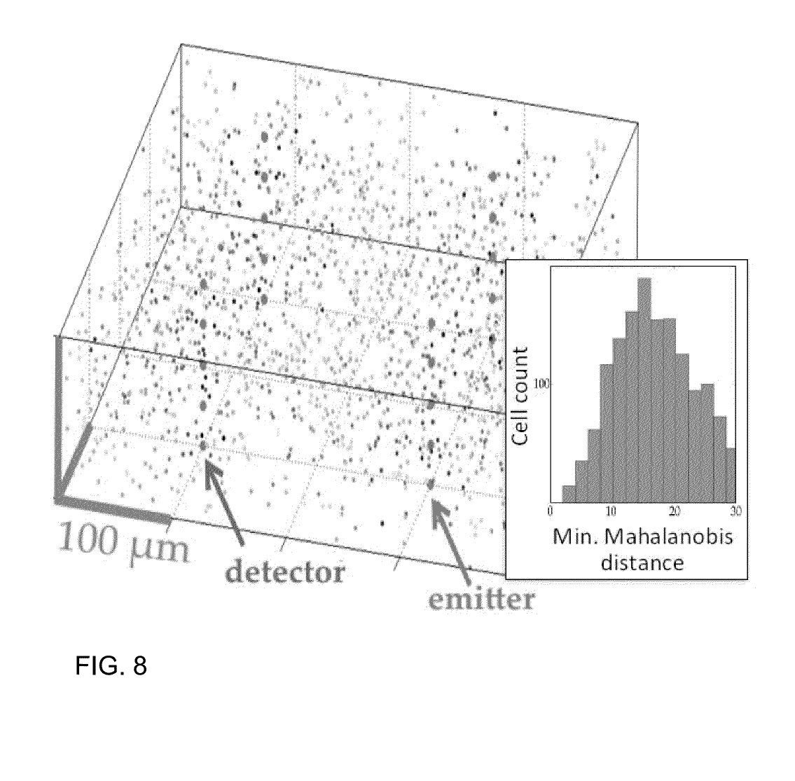

A simulation based on the prototype design for the neurophotonic probe arrays has been executed (FIG. 8). This demonstrates that the integrated neurophotonic probes described herein are capable of recording from very large populations of neurons, and can provide single-cell resolution. This modeling also shows that it is possible to assign calcium events that contribute the resulting, very-high-dimensional data stream to specific neurons. To validate the paradigm for optical spike sorting, a simulation involving 360 neurons randomly in a 282×282×50 μm³ unit volume (0.004 mm³) has been carried out. This mimics cell densities that are observed via two-photon imaging from the mouse visual cortex in vivo. The excitation intensity provided by each E-pixel to each neuron was determined, and the collection efficiency from each neuron to each D-pixel within one attenuation length was numerically calculated. Each specific combination of emitters and detectors yields an independent measurement (although not all such measurements in the resulting high-dimensional space are orthogonal); hence activity for each neuron is represented as point in the aforementioned space. To determine the separability of action potentials from neurons at different sites within the unit volume, the Mahalanobis distance between the activity of neuron pairs is calculated within this high-dimensional space. Modeling the data with Poisson photon statistics, the Mahalanobis distance is calculated assuming independent Gaussian noise and a variance equal to mean photon counts. The event isolation quality is then scored for each neuron by the minimum Mahalanobis distance to all other cells; this is the worse-case scenario for separability. Visualizing this data shows that neuron activity is separable throughout the volume, not solely near the neurophotonic probes (FIG. 8). The median Mahalanobis distance deduced is 17, with a 5% lower quantile of 7.1 and 95% upper quantile of 34. This confirms optical spike sorting can isolate events from different neurons; scaling these results suggests that a 25-shank neurophotonic probe array will be capable of recording the activity of 100,000 neurons with single-cell resolution.

### REFERENCES

The following publications are incorporated by reference herein:
[1] R. J. Douglas and K. A. Martin, Neuronal circuits of the neocortex. Ann. Rev. Neurosci. 27, 419-451 (2004).
[2] I. R. Wickersham, D. C. Lyon, F. J. Barnard, T. Mori, S. Finke, K. K. Conzelmann, J. A. Young, and E. M. Callaway, Monosynaptic restriction of transsynaptic tracing from single, genetically targeted neurons. Neuron 53, 639-647 (2007).
[3] W. Spooren, L. Lindemann, A. Ghosh, and L. Santarelli, Synapse dysfunction in autism: a molecular medicine approach to drug discovery in neurodevelopmental disorders. Trends Pharmacol. Sci. 33, 669 (2012).
[4] R. Delorme, et al., Progress toward treatments for synaptic defects in autism. Nature Medicine 19, 685 (2013).
[5] S. Siegert et al., Transcriptional code and disease map for adult retinal cell types. Nature Neuroscience 15, 487 (2012).
[6] W. Denk, J. H. Strickler, W. W. Webb, Two-photon laser scanning fluorescence microscopy. Science 248, 73-76 (1990).
[7] C. Grienberger, A. Konnerth, Imaging calcium in neurons. Neuron 73, 862-885 (2012).
[8] J. Akerboom, T. W. Chen, T. J. Wardill, L. Tian, J. S. Marvin, S. Mutlu, N. C. Calderon, F. Esposti, B. G. Borghuis, X. R. Sun, A. Gordus, M. B. Orger, R. Portugues, F. Engert, J. J. Macklin, A. Filosa, A. Aggarwal, R. A. Kerr, R. Takagi, S. Kracun, E. Shigetomi, B. S. Khakh, H. Baier, L. Lagnado, S. S. Wang, C. I. Bargmann, B. E. Kimmel, V. Jayaraman, K. Svoboda, D. S. Kim, E. R. Schreiter, L. L. Looger, Optimization of a GCaMP calcium indicator for neural activity imaging. Journal of Neuroscience 32, 13819-13840 (2012)
[9] G. Feng, R. H. Mellor, M. Bernstein, C. Keller-Peck, Q. T. Nguyen, M. Wallace, J. M. Nerbonne, J. W. Lichtman, J. R. Sanes, Imaging neuronal subsets in transgenic mice expressing multiple spectral variants of GFP. Neuron 28, 41-51 (2000)
[10] R. J. Cotton, E. Froudarakis, P. Storer, P. Saggau, A. S. Tolias, Three-dimensional mapping of microcircuit correlation structure. Frontiers in neural circuits 7, 151 (2013).
[11] B. F. Grewe, D. Langer, H. Kasper, B. M. Kampa, F. Helmchen, High-speed in vivo calcium imaging reveals neuronal network activity with near-millisecond precision. Nature Methods 7, 399-405 (2010.
[12] G. Katona, G. Szalay, P. Maak, A. Kaszas, M. Veress, D. Hillier, B. Chiovini, E. S. Vizi, B. Roska, B. Rozsa, Fast two-photon in vivo imaging with three-dimensional random-access scanning in large tissue volumes. Nature Methods 9, 201-208 (2012.
[13] M. Ducros, Y. Goulam Houssen, J. Bradley, V. de Sars, S. Charpak, Encoded multisite two-photon microscopy. Proceedings of the National Academy of Sciences (USA) 110, 13138-13143 (2013).
[14] A. Cheng, J. T. Goncalves, P. Golshani, K. Arisaka, C. Portera-Cailliau, Simultaneous two-photon calcium imaging at different depths with spatiotemporal multiplexing. Nature Methods 8, 139-U158 (2011).
[15] W. Mittmann, D. J. Wallace, U. Czubayko, J. T. Herb, A. T. Schaefer, L. L. Looger, W. Denk, J. N. Kerr, Two-photon calcium imaging of evoked activity from L5 somatosensory neurons in vivo. Nature Neuroscience 14, 1089-1093 (2011).
[16] C. Xu, In vivo three-photon microscopy of subcortical structures within an intact mouse brain, Nature Photonics (2013).
[17] J. M. Girkin, S. Poland, A. J. Wright, Adaptive optics for deeper imaging of biological samples. Current opinion in biotechnology 20, 106-110 (2009).
[18] M. Oheim, E. Beaurepaire, E. Chaigneau, J. Mertz, S. Charpak, Two-photon microscopy in brain tissue: parameters influencing the imaging depth. Journal of Neuroscience Methods 111, 29-37 (2001).
[19] INSCOPIX, located on the World Wide Web at inscopix.com.
[20] Mauna Kea, Inc., located on the World Wide Web at maunakeatech.com.
[21] M. L. Roukes, US patent applications (2011, 2012).
[22] M. L. Roukes, L. Moreaux, R. Cotton, A. Tolias, A. Siapas, US patent application (2013).
[23] M. L. Roukes and L. Moreaux, US patent disclosure (2014).
[24] A. Rochas, M. Gani, B. Furrer, P. A. Besse, R. S. Popovic, G. Ribordy, and N. Gisin, Single photon detector fabricated in a complementary metal-oxide-semiconductor high-voltage technology. Review of Scientific Instruments, 74 (7), 3263-3270 (2003).
[25] S. Tisa, F. Zappa, and I. Labanca. On-chip detection and counting of single-photons. In Electron Devices Meeting, 2005. IEDM Technical Digest. IEEE International. (2005).
[26] M. A. Marwick and A. G. Andreou. Fabrication and Testing of Single Photon Avalanche Detectors in the TSMC 0.18 μm CMOS Technology. In Information Sciences and Systems, 2007. CISS '07. 41st Annual Conference (2007).
[27] C. Niclass, M. Gersbach, R. Henderson, L. Grant, and E. Charbon, A Single Photon Avalanche Diode Implemented in 130-nm CMOS Technology. Selected Topics in Quantum Electronics, IEEE Journal of Quantum Electronics 13(4): p. 863-869 (2007).
[28] L. Pancheri and D. Stoppa. Low-Noise CMOS single-photon avalanche diodes with 32 ns dead time. In Proceedings of the 37th European Solid State Device Research Conference, ESSDERC 2007 (2007).
[29] M. Gersbach, J. Richardson, E. Mazaleyrat, S. Hardillier, C. Niclass, R. Henderson, L. Grant, and E. Charbon, A low-noise single-photon detector implemented in a 130 nm CMOS imaging process. Solid-State Electronics 53, 803-809 (2009).
[30] R. Field, J. Lary, J. Cohn, L. Paninsky, and K. L. Shepard, A low-noise, single-photon avalanche diode in standard 0.13 μm complementary metal-oxide-semiconductor process. Applied Physics Letters 97(21), (2010).
[31] R. Field and K. L. Shepard, A 100-fps fluorescence lifetime imager in standard 0.13-um CMOS, In Symposium on VLSI Circuits, 2013: Kyoto, Japan (2013)
[32] R. Field and K. L. Shepard, A 100-fps fluorescent lifetime imager in standard 0.13-um CMOS. IEEE Journal of Solid-State Circuits, invited, to appear (2014).
[33] H. Finkelstein, M. J. Hsu, S. Zlatanovic, and S. Esener, Performance trade-offs in single-photon avalanche diode miniaturization. Review of Scientific Instruments 78(10), 103103-5 (2007).
[34] A. Dalla Mora, D. Contirti, A. Pifferi, R. Cubeddu, A. Tosi, and F. Zappa, Afterpulse-like noise limits dynamic range in time-gated applications of thin-junction silicon single-photon avalanche diode. Applied Physics Letters 100 (24) (2013).
[35] J. T. Vogelstein, B. O. Watson, A. M. Packer, and R. Yuste, Spike Inference from Calcium Imaging Using Sequential Monte Carlo Methods. Biophysical Journal (2009).
[36] B. F. Grewe, Langer, D., Kasper, H., Kampa, B. M., Helmchen, F. High-speed in vivo calcium imaging reveals neuronal network activity with near-millisecond precision. Nature Methods 7 (5), 399-405 (2010).

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method for detecting functional cellular activity within a volume of a tissue, comprising
inserting a three-dimensional array of optical emitters and optical detectors into a volume of a tissue, the tissue volume comprising one or more cells labeled with an optical reporter of cellular activity,
illuminating the one or more cells with photons from the optical emitters of the three-dimensional array to generate optical signals from the optical reporter labeling the one or more cells, and
detecting the optical signals using the optical detectors of the three-dimensional array,
wherein the illuminating comprises one-photon excitation of the optical reporter.

2. The method of claim 1, wherein the optical signals are fluorescent optical signals.

3. The method of claim 1, wherein the tissue is nervous tissue or living brain tissue, and each cell is labeled with an optical reporter of neural activity.

4. The method of claim 3, wherein the optical reporter is a genetically encoded fluorescent protein, a chemical fluorescent reporter, or a fluorescent nanoparticle reporter, or a combination thereof.

5. The method of claim 1, wherein the array comprises elongated microsized shanks comprising the optical emitters and the optical detectors, each shank being about 100 μm or less in width.

6. The method of claim 5, wherein each shank comprises optical emitters and optical detectors.

7. The method of claim 5, wherein the shanks extend to any arbitrary location in the tissue.

8. The method of claim 1, wherein the optical emitters are time-gated.

9. The method of claim 1, wherein the optical emitters comprise optical elements for spatial profile control of the illuminating.

10. The method of claim 1, wherein the optical detectors comprise optical filters, focusing elements, planar optical elements, or metamaterial-based optical elements, or a combination thereof.

11. The method of claim 1, wherein the detecting comprises time-gated collection of the optical signals.

12. The method of claim 1, wherein the detecting comprises optical intensity sensing of the optical signals, or optical signal detection by avalanche current amplification of individual photon absorption events, or a combination thereof.

13. The method of claim 1, further comprising optical spike sorting of the detected optical signals.

14. A device for detecting functional cellular activity, comprising
elongated microsized shanks, each shank comprising one or more optical emitters and one or more optical detectors,
wherein the shanks are sized in width and thickness to fit between adjacent neuronal cell bodies in a neural tissue, and
wherein the shanks are arranged to form a three-dimensional array of the optical emitters and the optical detectors.

15. The device of claim 14, wherein the shanks are about 100 μm or less in width.

16. The device of claim 14, wherein the shanks are about 1 mm or more in length.

17. The device of claim 14, wherein the array has a pitch that is less than or equal to one optical attenuation length of a predetermined wavelength of light to be emitted from the optical emitters.

18. The device of claim 14, wherein the optical detectors comprise optical filters, focusing elements, planar optical elements, or metamaterial-based optical elements, or any combination thereof.

19. The device of claim 14, wherein the emitters are time-gated.

20. The device of claim 14, wherein the optical detectors comprise optical intensity sensors or avalanche current amplification sensors.

21. The device of claim 14, further comprising a time-to-digital converter connected to the optical detectors.
